(19) United States
(12) Patent Application Publication
(10) Pub. No.: US 2023/0039946 A1
(43) Pub. Date: Feb. 9, 2023

(54) Method and Device for Assisting and Enforcing a breathing process

(71) Applicant: Mikhail Levitin, Reeders, PA (US)

(72) Inventor: Mikhail Levitin, Reeders, PA (US)

(21) Appl. No.: 17/943,914

(22) Filed: Sep. 13, 2022

Related U.S. Application Data
(63) Continuation of application No. 16/852,492, filed on Apr. 19, 2020.

(51) Int. Cl. A61M 16/00; A61M 16/10; A61H 31/00; A61H 9/00; A61M 16/06; A61M 16/20

(52) U.S. Cl. CPC A61M 16/022; A61M 16/105; A61M 16/1075; A61M 16/0081; A61H 31/00; A61H 9/0092; A61M 16/06; A61M 16/201; A61H 2201/107; A61M 2205/8275; A61M 2202/0208; A61M 2230/432; A61M 2230/435; A61H 2201/1238; A61M 2205/07

(57) ABSTRACT

The present invention relates to the art of automatic regulation of pulmonary devices for imitating, assisting and/or enforcing the breathing process by converting Bag-Valve-Mask (BVM) or a similar device to enhance both phases of breathing: inhalation and exhalation while applying a variable pressure during the breathing process. It also replaces a mechanical chest compression to the sternum area for automatic pneumatic compression, and it could be complimented with the use of a Tens unit, can be used for extended period of time with a high level of reliability, simplicity, efficacy and low cost. The unique filtration system's goal in this invention is safety of the treating patient as well as assisting personnel. This portable and light device is recommended to be used as a resuscitator for the patients with mild to extremely suppressed or without respiratory drive. The source of power can be electrical, battery operated, manual or a combination thereof.

### THE DESCRIPTION OF PREFERRED EMBODIMENT

It will be understood that each of the elements described in present embodiment, or two or more together, may also find a useful application in other types, methods and constructions differing from the types described in this one.

When the invention has been illustrated and described as embodied in this Method and Device for Assisting and Enforcing the breathing process, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

### BACKGROUND OF THE INVENTION

A breathing process consists of two stages. The first stage of breathing, inhaling air into your lungs, is called inspiration (I) or inhalation. Inspiration happens because of a large breathing muscle called the diaphragm and muscles between the ribs contract, creating a negative pressure or vacuum inside the chest cavity.

When we take a breath, the air goes in through your nose and mouth and travels down your lungs (the first stage of a breathing cycle). The negative pressure draws the air that you breathe into your lungs.

The second stage of breathing, blowing air out of the lungs, is called expiration (E) or exhalation After the oxygen and carbon dioxide trade places in the alveoli, the diaphragm relaxes and positive pressure is restored to the chest cavity. This forces the used air out of the lungs, following the reverse of the path that it used to get in the lungs. The entire breathing process is repeated 10 to 30 times per minute depend on the condition of the person.

The present invention is designed to imitate the same physiological process by converting mechanical BVM into automatic one. Inspiratory time and expiratory time are then determined by portioning the respiratory cycle based on the set ratio. For instance, a patient with a respiratory rate of 10 breaths per minute will have a breath cycle lasting 6 seconds. A typical I:E ratio for most situations would be 1:2, if we apply this ratio to the patient above, the 6-second breath cycle will break down to 2 seconds of inspiration and 4 seconds of expiration. Increasing the I.E ratio to 1:3 will result in 1.5 seconds of inspiration and 4.5 seconds of expiration Thus, a “higher” l:E ratio results in less inspiratory time and more expiratory time in the same length of the breath cycle. In addition, we can regulate the I:E ratio with the power of the air flow during inspiratory and expiratory time.

Patients with lung failure are often prescribed mechanical ventilation as a lifesaving intervention while waiting the recovery of the patient's own lungs, or as a bridge to lung transplantation. The currently available technology for ECMO or for high frequency ventilators requires large very bulky and expensive machines that practically have a selective usage. The large, heavy nature of these machines is not user friendly as well. These devices are not truly portable and require the assistance of another person, often the presence of trained, licensed specialists to be physically available around the clock to ensure that these systems are functioning properly or is involved the administrative physical activities that has a limit due to exhaustion.

The need for near constant supervision by a specialist greatly reduces their ability to spend time with multiple patients that is critical during an intensive care.

Hospitals across the US and around the world brace for cases of the novel coronavirus, departments in charge of caring for the sickest patients are grappling with how they are going to respond.

By some estimates, millions of Americans sickened by coronavirus might need a stay in an intensive care unit, the part of the hospital devoted to providing advanced life-saving care. That will likely put a strain on staff, supplies of equipment like ventilators and put facilities at enormously high mortality risk

Although invasive mechanical ventilation saves tens of thousands of lives each year, it can also be harmful, causing or worsening acute respiratory distress syndrome (ARDS) when misapplied. The repetitive stretching of lung tissue during positive pressure ventilation can damage fragile alveoli already made vulnerable by preexisting illness. This potentially lethal process has been called ventilator-induced lung injury. That is why the emphasis should be moved on the second stage, exhalation, to remove phlegm and excessive amount of CO2. If carbon dioxide levels are allowed to accumulate without disposal, the blood will become more acidic, leading to cellular damage on a systemic scale, which may ultimately lead to organ failure or death.

The respiratory problems in COVID-19 patients, especially in intensive care units, have recently been largely attributed to micro-vascular thrombosis, which leads to decreased blood flow to the tissues, with possible infarcts. Micro-vascular thrombosis is caused by clotting of the red blood cells and it has a similar effect as the Rouleau effect. Several publications have reported that the Rouleau effect promotes blood clotting of red blood cells. Clotting contributes to oxygen deficiency and respiratory problems and is responsible for thrombosis in COVID-19 patients

The patients with COVID-19 often have what seems to be a pervasive but initially overlooked feature of “silent hypoxia”. Unlike many other respiratory diseases, COVID-19 can slowly starve the body of oxygen without initially causing much shortness of breath. By the time some patients have trouble breathing or feel pressure in the chest, among the symptoms the U S Centers for Disease Control and Prevention lists as emergency warning signs, they are already in life threaten condition.

Thus, there is a dramatic need in medicine for a portable, reliable, simple and inexpensive integrated breathing assisting automatic device which does not require specially trained personnel to operate it and can also help by releasing medical personnel from constant patient supervision for additional vital tasks.

Further, there is a need for such a device that can imitate physiological breathing stages, does not require significant financial investment, especially in critical ambulance situations, while highly increasing the number of patients that requested a critical care, eventually saving more lives. The simplicity, low-cost and ease of production of said device, and the fact that it can be made within a short time ought to put it in great demand in any health care system. The present invention addresses this unmet need in medicine.

### SUMMARY OF THE INVENTION

In one embodiment, a portable device for assisting and/or enforcing breathing process, includes a compressor with a variable negative or a variable positive air pressure, having filters to disinfect the air coming to/from the patient through a Bag-Valve-Mask (BVM), which operated in a rigid durable pressure chamber to distribute air flow through an outlet to or from the nose and/or mouth of the patient with extremely suppressed or without respiratoly drive. More specifically, a preferred embodiment of the present invention includes an engine controlled a compressor which regulates series of variable repeated portions of positive pressure followed by series of variable repeated portions of negative pressure supply to durable pressure chamber with BVM that allow the air to flow to/from the patient with mild to extremely suppressed or even without respiratory drive to imitate natural physiological breathing cycles.

According to the second embodiment of the present invention a compressor with a variable negative or a variable positive pressure provides air flow to the BVM inside durable pressure chamber, inflates or deflates the bag (BVM) and moves the air flow in a filter to disinfect the air coming to/from the nose and/or mouth of the patient with extremely suppressed or without respiratory drive, and an energy support system feeds said compressor and an electromagnetic coil with a tiler in it and which regulates frequency, amount of flow and depth of the patient's breathing. The air flow delivered to the patient is passing an adjustable in length electromagnetic heater to deliver preheated air to the patient for some therapeutic benefits. The heater is a device using the electromagnetic induction principle to convert the electric energy into heat energy. The induction heater turns the alternating current of 110V. 220V or the three-phase AC 380V, 50/60 HZ into DC, then turns the DC into the high frequency 1˜40 KHz high current electricity used for heating the breathing air.

According to the third embodiment of the present invention a variable positive pressure from said compressor affects said rigid durable pressure chamber and without a physical contact with the ball compresses BVM forcing the air to flow from BVM through an inlet to the patient to initiate an inhalation action while a positive pressure valve is opened and a negative pressure valve is closed. In turn, when a positive pressure is off the negative pressure valve is opened and negative pressure inside said durable pressure chamber remains variable and through an outlet engages an exhalation process as a result of expending of said ball of said BVM. When a positive pressure exceeds the maximum preset level or negative pressure plunges below minimum preset level a control valve temporally opens to normalize the pressure inside the pressure chamber to the preset comfort pressure zone.

According to the fourth embodiment of the present invention a combination of the first and second embodiment is set up as one process (inhalation) follows another (exhalation) in repetitive variable portions supply of negative pressure followed by repetitive variable portions supply of positive pressure in cycling motions.

According to the fifth embodiment of the present invention the air flow alternated by gas to or from the nose and/or mouth of the patients passing through a filtration unit consisted of a coil unit with Extremely Low Frequency of Electromagnetic Field (ELFEF) (380-480V/m, 120-140 nT1) 53-60 GHz embraced over a thermo filtration tube with a fiber rope inside, which is lubricated with an antibacterial liquid (for example, hydrogen peroxide) and saline on both sides of said rope and an engine to move said rope to a collecting container for used filter-rope on one side of said rope when the both sides of the rope are connected to create a closed loop for a micro current flow induced by electromagnetic field of said coil to affect viruses infectious ability.

According to the sixth embodiment of the present invention the compressor produces an air flow that pushes an upper valve in the pump in closing position and presses movable membrane against spring while air flow is produced by said membrane which closes on its way opened valves and delivers air through the outlet to nose and/or mouth of said patient. When air pressure from said compressor stops, the upper valve gets open by gravity and by the force of said membrane which is pushed back by it's spring, emptying the part of the pump from air into opened outlet and at the same time sucking air from said patient into opened outlet.

According to the seventh embodiment of the present invention the air flow from said compressor with variable levels of positive/negative pressure air connected through said pipe to BVM which regulates time and pressure of supplying portions of the air from or to a patient while the same or another compressor inflates a chest cuff on the patient's chest on the level of the sternum to compliment the exhalation phase of the patient's breathing, the cuff is deflated when the inhalation is started.

According to the eighth embodiment of the present invention the exhalation phase of the breathing is accommodated with the topical electrical stimulation by a TENs unit applied to the patient's abdominal and intercostal muscles.

According to the ninth embodiment of the present invention the air flow from said compressor with variable levels of positive/negative pressure air connected through said pipe to an inlet of said monitor which regulates time and pressure of supplying portions of the air from outlet through a pipe to a chest cuff on the patient's chest on the level of the sternum to accommodate the exhalation phase with the additional compression by the chest cuff and/or this process complemented with TENs unit.

According to the tenth embodiment of the present invention the air flow from said compressor with variable levels of positive/negative pressure air flow at the preset rate I/E and E/I, with the composition of sweep gas, including oxygen and/or carbon dioxide ratio, lipid peroxidation (LPO) due to neutrophil-derived reactive oxygen species, and the temperature of the air flowing through the filter set to better assist the breathing process for the patient and to prevent or reduce the potential for causing adverse health effects or for a medically supervised increase of core temperature to eradicate viral colonies or clusters.

### DETAILED DESCRIPTION

It is to be understood that the figures and descriptions of the present invention have been simplified to illustrate elements that are relevant for a clear understanding of the present invention, while eliminating, for the purpose of clarity, many other elements found in the field of artificial lungs or lung assist devices for individuals without respiratory drive. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in testing the present invention, the preferred materials and methods are described herein.

It is also to be understood that the terminology used herein is for the purpose of describing particular embodiments only, and is not intended to be limiting.

The terms “patient,” “rope,” “engine,” and the like are used interchangeably herein, and refer to any animal amenable to the systems, devices, and methods described herein. Preferably, the patient, subject or individual is a mammal, and more preferably, a human.

The description of “positive” pressure means that it is above the ambient one “negative” pressure means below the ambient pressure.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 8 should be considered to have specifically disclosed sub-ranges such as from 1 to 3, from 1 to 4, from 1 to 5, from 2 to 4, from 2 to 6, from 3 to 8 etc., as well as individual numbers within that range, for example, 1, 2, 2.7, 3, 4, 5, 5.3, . . . and 8. This applies regardless of the breadth of the range.

The present invention relates to the art of automatic pneumatic regulation of pulmonary devices for assisting and/or enforcing the breathing process which can be used for extended periods of time with a high level of reliability, simplicity and efficacy not by a specially trained technician, but by volunteers, family members or by a patient. The following is a detailed description of the best presently known mode of carrying out said invention. This description is not to be taken in a limiting sense, but is made merely for the purpose of illustrating the general principles of the invention. The scope of the invention is defined by the appended claims.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is an illustration of the method of assisting in breathing that provides a variable positive or a variable negative pressure from a compressor 1 by inlet 2 to a rigid durable pressure chamber (DPC) 3 with BVM 4 inside it, and that effects the air flow in the filtration system 7 through a filtration tube 7.1 inside an electromagnetic coil 7.2 that embraced the filtration tube 7.1 with a fiber rope 6 in it and two electrically connected by a conductor 6.1 ends of the rope having containers with saline and hydrogen peroxide solution in each one and the fiber rope is puled by engine 12, which movement is synchronized by a monitor 9.1 with inhaled and exhaled air by a patient through an inlet 8.1 and said air is moved into a quarantine container 11 while the air flow is circulated in a heating tube 7.3 to and out of a mask attached to a nose and/or mouth of a patient 8 according to one embodiment.

FIG. 2 is an illustration of the method of assisting in breathing that provides a variable positive or a variable negative pressure inside a rigid durable pressure chamber 3 with BVM 4 inside it, and that effects the air flow in the filtration system 7 through a filtration tube 7.1 inside an electromagnetic coil (EMC) 7.2 that is charged by a power support system 9 and that embraced the filtration tube 7.1 with a fiber rope 6 in it and two electrically connected by a conductor 6.1 ends of the rope having containers with saline and hydrogen peroxide solution in each one and the fiber rope is puled by engine 12 into a quarantine container 11 while the air flow is circulated in a length changeable heating tube 7.3 to and out of a mask attached to a nose and/or mouth of a patient 8 according to one embodiment.

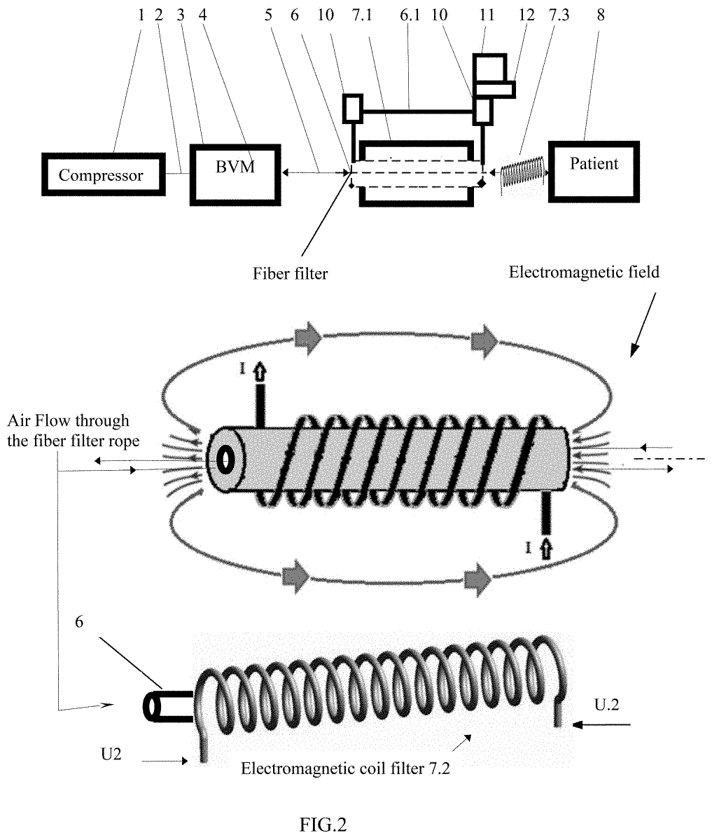

FIG. 3 is an illustration of the method of assisting in breathing with the air flow from said compressor 1 that is electrically supported with the power support system 9 with voltage U1 (110V; 220V) and through said pipe 2 with variable levels of compressed air connected to the rigid durable pressure chamber 3 with BVM 4 inside, which operated in said durable pressure chamber 3 a under variable positive/negative pressure without a mechanical contact with BVM 4 to assist a breathing process for the patient 8 through an inlet 7.3 to a filter 7, and where a valve 17 regulates positive—negative pressure supply in turn to avoid an arrow if it provided simultaneously and valve 22 of the outlet 16 opens when a positive pressure exceeds the maximum preset level or negative pressure plunges below minimum preset level to normalize the pressure inside the pressure chamber 3 within preset comfort pressure zone.

FIG. 4 is an illustration of the method of assisting in breathing that provides a variable positive or a negative pressure from a compressor 1 by inlet 2 inside a rigid durable pressure chamber 3 with BVM 4 inside it, and that effects the filtration in said system 7 by providing the electrical charge from said power support system (PSS) 9 to said EMC 7.2 (U2=10 kv/cm) with the filtration tube 7.1 and a fiber rope 6 in it inside EMC 7.2 and two electrically connected by a conductor 6.1 ends of the rope 6 having containers 10 with saline and hydrogen peroxide solution in each one and the fiber rope is puled by an engine 12 into a quarantine container 11 while the air flow is circulated by a tube 7.3 to and out of a mask attached to a nose and/or mouth of a patient 8 and the filtration is achieved by passing air to/from the nose and/or mouth of said patient 8 to kill pathogens with unstable electrically charged atoms and pathogens unfriendly solution according to one embodiment.

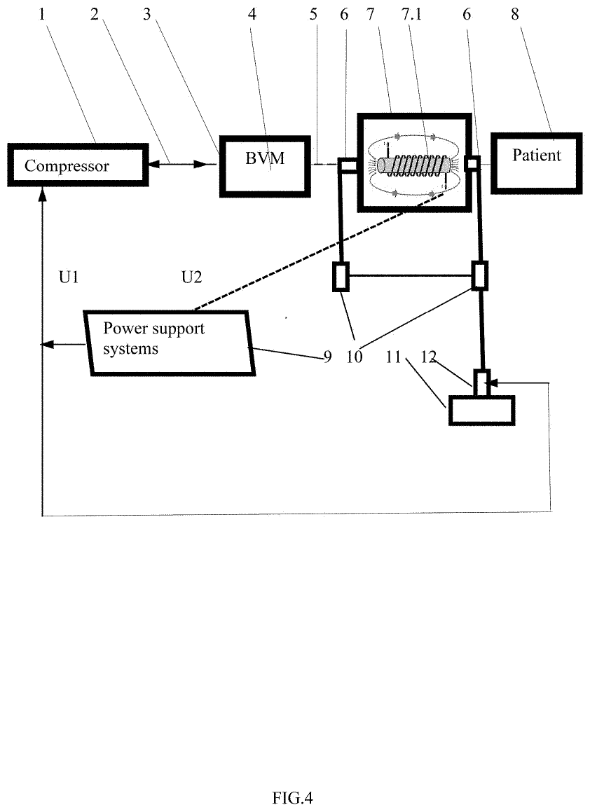

FIG. 5 is an illustration of the method of assisting in breathing that provides a variable positive or a negative pressure from a compressor 1 by inlet 2 to a rigid durable pressure chamber 3 with BVM 4 synchronized with a complimented topical stimulation abdominal and intercostal muscles by transcutaneous electrical nerve stimulation unit (TENS) 14 to enhance an exhalation phase of the breathing and TENS is off during an inhalation phase.

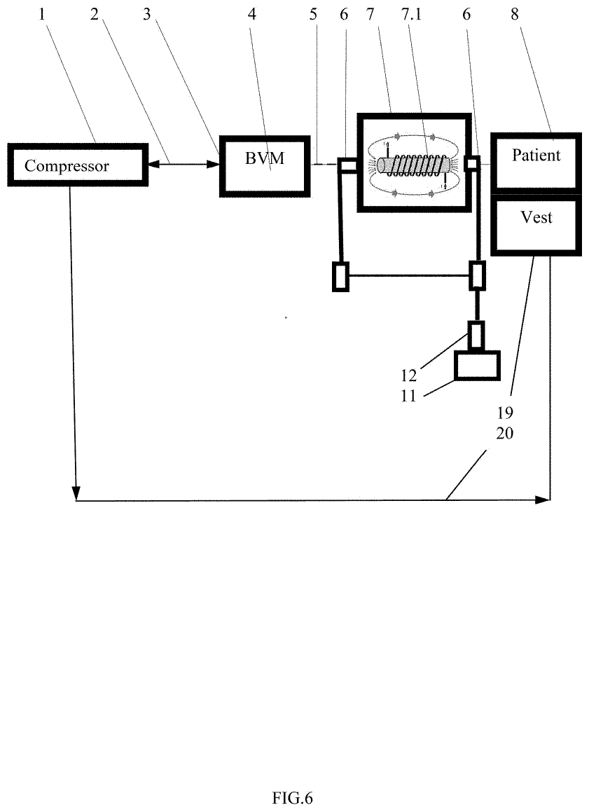

FIG. 6 is an illustration of the method of assisting in breathing that provides a variable positive or a negative pressure from a compressor 1 by inlet 2 to a rigid durable pressure chamber 3 with BVM 4 synchronized at an exhalation phase of the breathing with a complimented topical stimulation sternum of the embraced chest of the patient by a pneumatic vest 19 that gets inflated by the pressure from the compressor 1 by outlet 20 to enhance an exhalation phase of the breathing and gets deflated during an inhalation phase.

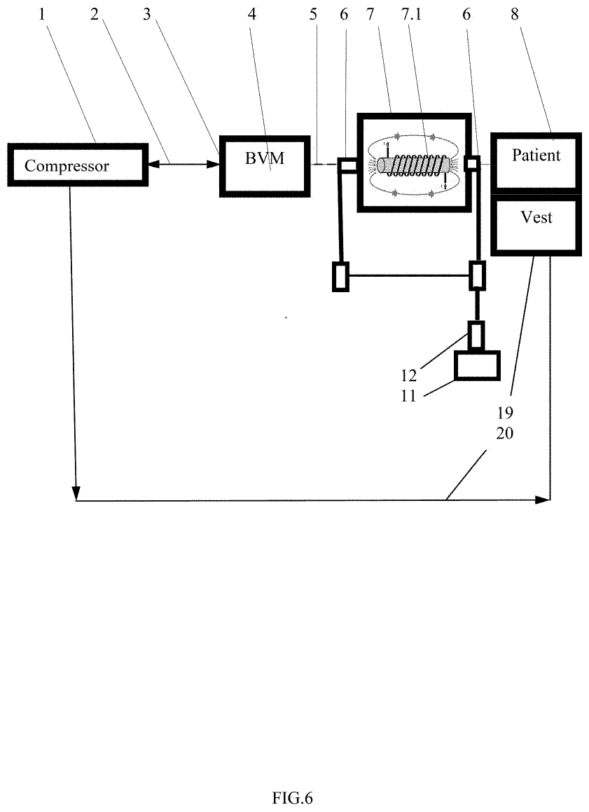

FIG. 7 is an illustration of the method that provides a variable positive or a negative pressure from a compressor 1 to produce directly an air flow through an inlet 2 into a rigid durable pressure chamber (DPC) 3 where it reposition a movable wall (MV) 21 in 620 the DPC3 divided by said MV 21, and is moving sliding valves 23, that are firmly attached to the MV 21, in a closed position and said MV 21 delivers air through the outlet 7.1 to the nose and/or mouth of said patient 8 through the filtration system 7,35 or a combination of both. It imitates the inhalation phase of the breathing. When air pressure changed to a negative, the sliding valves 22 get opened to expel the air from said DPC and from the patient according to the breathing process I/E ratio.

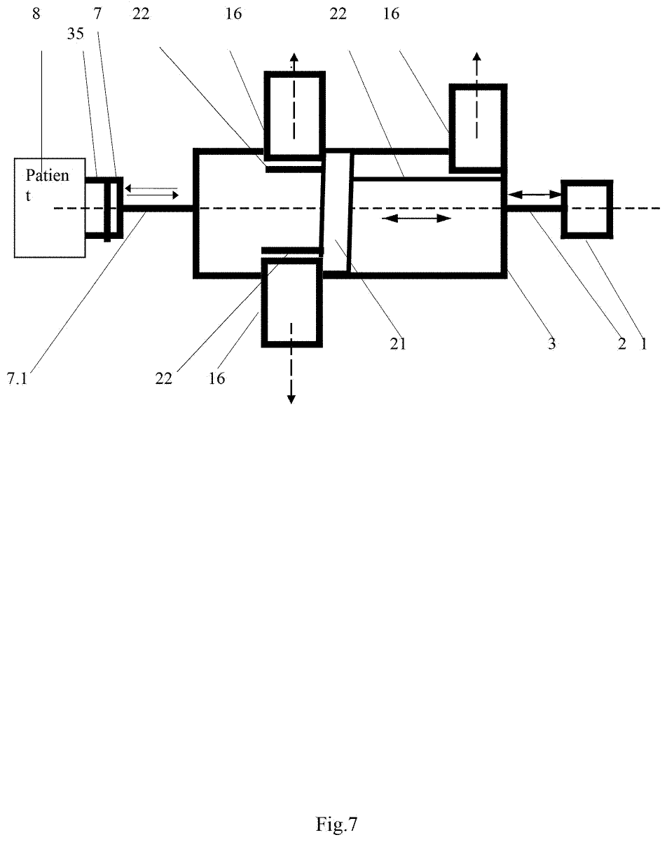

FIG. 8 is an illustration of the method in assisting breathing that provides a filtration of the air flow from or to the patient 8 when the air flow passing through electrically charged by PSS 9 electrode 24 that rotates around it's axis 25 by the breathing air flow that is pushing wings 23, firmly set on said electrode 24, clock wise or counter clock wise and brushes 26 that are firmly set on said electrode touching contacts 27 inside of the filter chamber 28 and creating sparks that disable pathogens in the air.

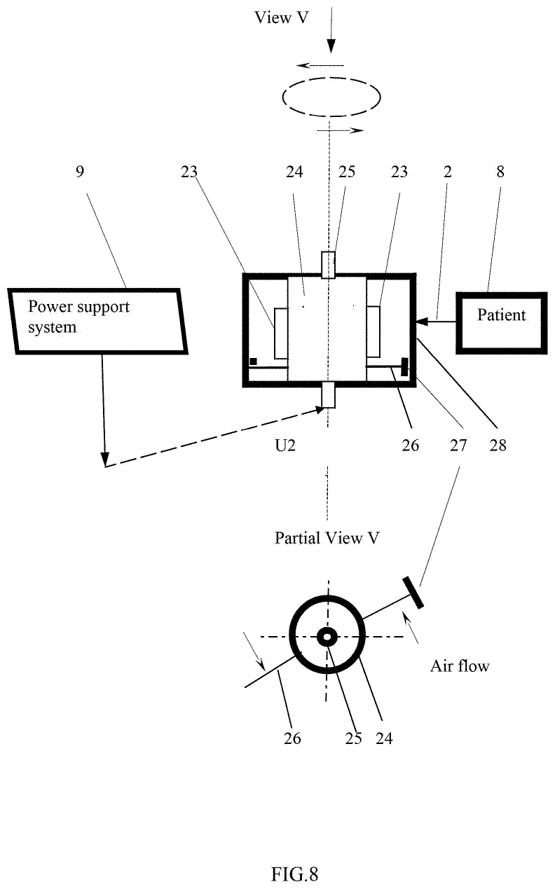

A process/method claim is directly infringed only if each step of the claimed method is performed. In NTP, Inc. v. Research in Motion, Ltd.,22 (NTP) the Federal Circuit rejected claims of method use infringement. A process or method is merely a sequence of actions, such a claim is infringed only when every operative step is performed.

A system comprises multiple distinct components that are effective only when used as a whole. Centillion Data Systems, LLC v. Qwest Communications Int'l, Inc., the court decided that use of a system required the infringing party to put the invention into service, i.e., control the system as a whole and obtain benefit from it.

The above stated facts are directly related to the U.S. Pat. No. 2,284,964A; 5195527A, 6209540B1; 20040022669A1; 20210259343A 1.

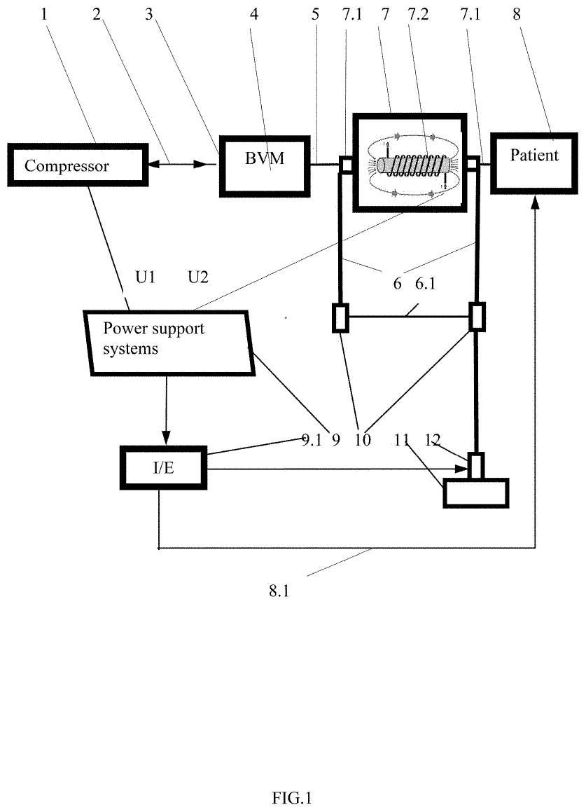

Particular Mautz, U.S. Pat. No. 2,284,964A, is focusing on the assisting in automatic inhalation to deliver medications during anesthesia and “being used merely as an anesthetizing machine, the bag 12 performs the function of the usual breathing bag and the bag contracts and expands in the usual manner in accordance with the patients respiration” while using permanent positive pressure. “During exhalation air flows through the pipe 4 illustrates our improved respirator apparatus in more or less diagrammatic form, and in which—FIG. 1 is a diagrammatic layout of our improve respirator attached to an anesthetizing machine.” The exhalation process occurs naturally.

The rest of the mention inventions are using permanent positive or negative pressure before it is changed to each other.

All these inventions have been applied before the pandemic and have serious problems when we started to use these methods on a high scale basis.

According to “Control of Breathing and Acute Respiratory Failure” by Robert K. Kanter, in Pediatric Critical Care (Third Edition), 2006: “Large negative intratracheal pressure with forced inspiration worsens upper airway obstruction, whereas positive intrapleural pressure during forced expirations can cause collapse of intrathoracic airways. Dynamic airway obstruction in the dyspneic child may account for rapid progression of respiratory failure in some cases.”

Res Biomed Eng. 2022; 38(1): 267-278. Published online 2021 Apr. 22. doi: 10.1007/s42600-021-00149-0 PMCID: PMC8060160:

“The role of a negative pressure ventilator coupled with oxygen helmet against COVID-19: a review” by Kathiresan Chandrasekaran and Aadharsha Monikandan Shaji:

“The coronavirus (SARS-COV-2) pandemic has provoked the global healthcare industry by potentially affecting more than 20 14 million people across the globe, causing lasting damage to the lungs, notably pneumonia. ARDS (acute respiratory distress 15 syndrome), and sepsis with the rapid spread of infection. To aid the functioning of the lungs and to maintain the blood oxygen 16 saturation (SpO2) in coronavirus patients, ventilator assistance is required.”

“Positive Pressure Ventilation” by Iordan Potchileev; Maksym Doroshenko; Asif N. Mohammed. May 15, 2022:

“Positive pressure ventilation is a form of respiratory therapy that involves the delivery of air or a mixture of oxygen combined with other gases by positive pressure into the lungs. As gas enters the lungs, the inter-alveolar pressure increases until a change in flow or pressure are detected by the machine delivering the mixture, or the set volume of gas was delivered to signal the end of a breath.”

“Barotrauma occurs when there is alveolar damage due to high pressures entering the lungs. Specifically, the transalveolar pressure, which is the difference in pressure between the alveolus and the surrounding interstitial space, is increased to such an extent that the epithelial lining of the alveoli is damaged. With repeated breaths and inappropriate ventilation, the damage often occurs on a microscopic level until it is severe enough to cause an overt pneumothorax, subcutaneous emphysema, or pneumomediastinum, which are all conditions associated with high mortality rates.”

The above stated problems are avoided with the present invention due to the fact that positive and negative pressure delivered in variable series of supply before it was switched to each other that provide a unique treatment process. In this case intratracheal pressure does not last long enough to force upper airway obstruction and barotrauma to alveols. The new physiological result has been achieved that allowed to avoid fatalities.

There is no infringement if the product of the process has been “materially changed by subsequent processes” or if “it becomes a trivial and nonessential component of another product.”

This provision is rooted in antiquated views of methods and processes. The legislative history confirms this view of process patents, such as when then—Commissioner Mossinghoff noted that “[a] process patent, however, only protects a process or method of making an article or product.” Section 271(g)'s safe harbor also reflects this outdated view of processes: only a physical product would seem to be able to be materially changed or a trivial and nonessential component.

The unique innovative feature of presented invention is the idea of enhanced exhalation and inhalation with variable level of positive and negative effectively filtrated air pressure.

By depressurizing the chamber faster than it depressurizes in the atmosphere, we can overcome the material limitations of the bag valve mask and create a safe pressure gradient within the intubated patient that enhances exhalation, which no other device can offer.

This principle has never been researched or pursued academically besides the Iron Lung devices in the early 20th century and can potentially be very important for alleviating acute respiratory distress syndrome (ARDS).

The U.S. Pat. No. 3,683,655A by White, “Breathing assist apparatus” relates to the device while our invention is the method. It is important to outline that White is using his device as a part of active modality when “the pressure is applied during the exhalation phase of the breathing cycle and the pressure applying means is activated by the person's efforts to exhale” and “applying pressure to the abdomen and lower ribs over a time period equivalent to the exhalation phase in breathing”.

The present invention is provided a pressure to the different part of the body, “chest cuff”, the mid chest, the sternum, were the applications recommended by CPR instructions, with a different physiological effect than it is done in the present invention with a different medical goal to expel CO2 with phlegm.

According to the above explained reasons U.S. Pat. No. 3,683,655A diverges from the present invention.

The U.S. 20020188332A1 by Lurie relates to “devices and methods of electrically stimulating the phrenic nerve. In one embodiment, electrodes are placed posterior and anterior in the region of the cervical vertebrae. Electrical current having a multi-phasic waveform is periodically applied to the electrodes to stimulate the phrenic nerve, thereby causing the diaphragm to contract“while stimulating the internal muscle of the body.” This method required a surgical manipulation, stimulates internal muscle-diaphragm, and should be performed by a qualified physician in medical environment.

Phrenic nerve stimulation, also known as phrenic nerve pacing, has been used to restore some form of breathing function in patients with respiratory paralysis resulting from spinal cord injuries or from neurological disorders such as congenital central hypoventilation syndrome, central sleep apnea, and diaphragm paralysis.

The phrenic nerve is a bilateral, mixed nerve that originates in the neck and descends through the thorax to reach the diaphragm. It is not connected with abdominal and intercostal muscles and an electrical signal from this nerve will not be able to stimulate said muscles.

Present invention is related to the topical stimulation of the abdominal and intercostal muscles which is simple, safe, user friendly and does not require a special environment.

According to the above explained reasons, U.S. 20020188332A1 diverges from present invention.

The U.S. 20040060558 by Gradon, is “designed to provide both temperature and flow rate sensing of the gases flow by incorporating two sensors (preferably thermistors) and the shape and alignment of the probe enables accurate readings by reducing the occurrence of condensation on the sensors. A number of possible applications are disclosed wherein the flow sensor is included in humidification control systems which provide a patient with a desired humidity level or simplify the amount of user input required or wherein the flow sensor provides a controller with flow information which may then be used to determine certain, possibly dangerous, conditions”. Another way, Gradon changes breathing air conditions by regulating temperature through humidity. This is the invention for a device.

In the presented invention we have a method of regulating temperature through the breathing air conditions by the length of the heating unit and an inhalation/exhalation ratio.

According to the above explained reasons, US Patent 20040060558 by Gradon diverges from our technical solution.

The U.S. 20050021102A1 by Ignagni is “the method can include the steps of implanting an electrode adjacent a target site in the diaphragm of the patient and operating the electrode to deliver a sufficient amount of electrical stimulation to the target site in the diaphragm of the patient to cause the diaphragm to contract.” It affects the internal muscle by “implanting an electrode adjacent a target site in the diaphragm of the patient”. The diaphragm is an internal muscle that is not reachable by an external electrical applications. It can be done only in medical facilities by a medically trained professionals.

The phrenic nerve is a bilateral, mixed nerve that originates in the neck and descends through the thorax to reach the diaphragm. It is not connected with abdominal and intercostal muscles and an electrical signal from this nerve will not be able to stimulate said muscles.

Present invention in the opposite, presents the topical method of stimulation of the abdominal and intercostal muscles which is more effective, safe and user friendly.

According to the above explained reasons, U.S. Pat. No. 5,485,754, diverges from the present invention.

The U.S. 20080056934A1 by Taui, relates to “a system for treating air and processing materials, comprising: at least one diffusive plasma reactor, each diffusive plasma reactor having insulated electrodes and a reaction chamber defined between the electrodes; a diffuser located in the reaction chamber between the electrodes; and a power supply for supplying high voltage alternating current to the electrodes; wherein the electrodes generate plasma within the reaction chamber to treat air passing through the reaction chamber or process materials placed in the reaction chamber.”

In the present invention a spark with an electrode is engaged by breathing air flow with no plasma.

According to the above explained reason, US Patent U.S. 20080056934A1 diverges from the present invention.

The U.S. 20080230064A1 by Tham, relates to “a method of setting inspiratory time in pressure controlled mechanical ventilation sets a subject's inspiratory time“and” . . . giving patient to normally exhale”.

In the present invention the therapeutic target is the expiratory time, forcing patient to exhale (not “normally”) to eliminate CO2 and phlegm.

According to the above explained reasons, U.S. Patent U.S. 20080056934A1 diverges from our technical solution.

The U.S. 20090205663A1 by Vandine, relates to producing “a drug cloud and which can then be injected into a spacer where it can be inhaled by a user.”

The present invention is related to the method of complete imitation of a breathing process.

According to the above explained reasons, US Patent U.S. 20090205663A1 diverges from our technical solution.

The U.S. 20120272960 by Milne, relates to “breath delivery synchronization” and more particular to “A method for ventilating a patient with a ventilator, comprising: monitoring a physiological parameter of the patient based on one or more received sensor measurements; calculating a first derivative of the physiological parameter for a first sample period; updating a sample count to form a first updated sample count for the first sample period based on first comparison results from at least one of the following: comparing a first trigger count threshold to a previous trigger count threshold selected in a previous sample period, comparing the first derivative to a previous first derivative calculated for the previous sample period, and comparing the first derivative to a first level; comparing the first updated sample count to the first trigger count threshold; and triggering inspiration based on a first result of the comparing of the first updated sample count to the first trigger count threshold.”

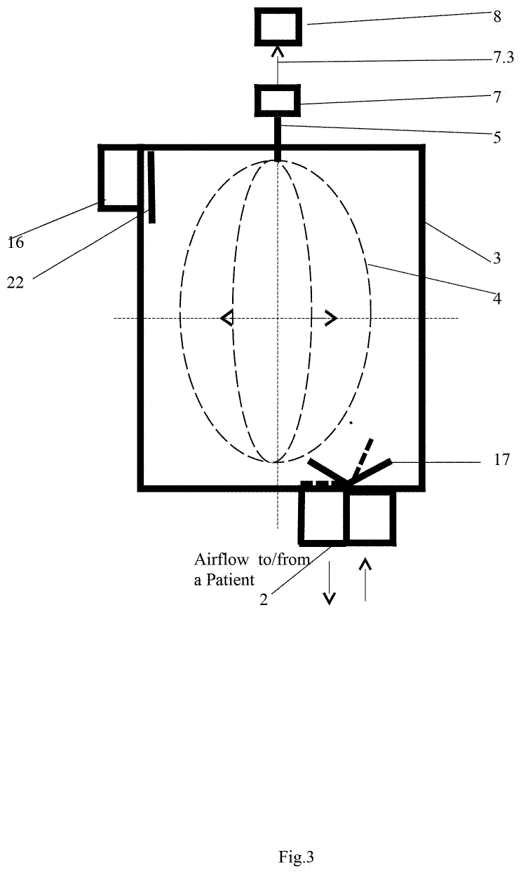

And additional note “Method 200 also includes a calculating operation 204. The ventilator during the calculating operation 204 calculates the first and/or second derivative of the physiological parameter for each sample period. In some embodiments, the ventilator during the calculating operation 204 performs an amplification operation 204a, as illustrated in FIG. 3. The ventilator during the amplification operation 204a of the calculating operation 204 amplifies the first and/or the second derivative of the physiological parameter. In some embodiments, the ventilator during the amplification operation 204a of the calculating operation 204 amplifies the first and/or second derivative by 5, 10, 20, 30, 40, 50, 60, 70, 80, and/or 90”.

This method is “triggering inspiration based”, extremely costly, takes time and requires specially train personnel and vulnerable to algorithm errors in the calculations.

In the present invention the focus is on the expiration process, which allow us to efficiently expel CO2 and phlegm and provide an optimum amount of 02 to the patient.

The method is simple and user friendly. In present invention used different type of ratios for different purposes.

According to the above explained reason, US Patent 20120272960 diverges from the present invention.

The U.S. 20130061849A1 by Lemper, relates to the device that can improve an inhalation of medications through the nebulizer, “breathing apparatuses, and methods for administering a solution by inhalation to a patient.”

In the present invention the focus is on the method to effectuate the exhalation process, which allows us to efficiently expel CO2 and phlegm and improve the efficacy of the whole breathing process for a patient.

According to the above explained reasons, US Patent 20130061849A1 diverges from our technical solution.

The U.S. Pat. No. 8,534,282B2 by Bergman, relates to a mechanical device where “a mechanical compression squeezer for cyclically squeezing said squeeze bag from its outside and releasing said squeeze bag for expansion”.

On the contrary, the present invention is about the method and it does not suggest any mechanical contact with Ambu-Bag. Our method is based on a pneumatic method of the operation without any mechanical contact. According to the above explained reasons, U.S. Pat. No. 8,534,282B2 diverges from present invention.

The U.S. 20190143154A1 by Gangadhar, relates to “a rolling mechanism may rotate the roller, allowing for a fresh section of filter media to be placed in the air path of the respirator. The rolling mechanism may be controlled automatically by a motor and/or manually.” And more particular, “a filter media configured to filter harmful substances from the air passing through the respirator, wherein the filter media comprises a thin sheet of filter media”.

In the present invention the filter is an electrically charged rope which is placed along with the air flow and has a different filtration mechanism. The moving filter mechanism is synchronized with inhaled and exhaled air by a patient.

According to the above explained reasons, U.S. 20190143154A1 diverges from the present technical solution.

The U.S. 20190336713A1 by Piracha relates to a mechanical device where “the actuator has a convex contact surface that compresses the bag” and more “wherein the bag has a longitudinal axis, and the convex contact surface is configured to extend along the longitudinal axis.”

In the present invention we avoid any mechanical contact with Ambu-Bag and the method is based on a pneumatic operation without any mechanical contact of the bag. While providing pneumatic affect to each and every point of the bag we can gain much more effective and precised control of the air flow to and out of the patient.

According to the above explained reasons, US Patent 20190336713A1 diverges from the presented invention.

The U.S. 20200306472 by Modi is an invention for a device that is “a patient ventilator system includes a patient delivery circuit having an inspiratory section that delivers an inspiratory gas flow to a patient and an expiratory section that receives expiratory gas flow from the patient, wherein a bidirectional blower motor drives the inspiratory gas flow in the inspiratory section and controls the expiratory gas flow in the expiratory section.”

While Modi's idea to” . . . effectuate the inspiratory and expiratory gas flow” and invention is focused on the “potential for causing adverse health effects or for a medically supervised increase in core” that is absolutely different medical goal.

Also Modi changes constant negative pressure for constant positive pressure and so on that can potentially give serious side effects explained above.

In the present invention the method is used to effectuate the exhalation process, which allows to efficiently expel CO2 and phlegm and improve the efficacy of the inhalation to the patient by providing series of variable negative followed with series of variable positive pressure applications and following so on. It helps to avoid side effects coursed by Modi's method and saves lives.

According to the above explained reason, US Patent 20200306472 diverges from the presented technical solution.

The U.S. Pat. No. 5,195,527 to Hicks is the “invention relates to an improved respiratory filter included in a respiratory system of the kind used in anesthesia and/or patient ventilation. Such filters may be used both for bacteriological control and as heat and moisture exchangers”. In the present invention under the filtration issue presented the method of electric sparks and a thermo heating unit were the filtration related to the length of the unit and different method of creating an electric spark involved.

According to the above explained reason, U.S. Pat. No. 5,195,527 to Hicks diverges from the present invention.

It is known that in order to arrive at a claimed invention by modifying the references the cited art must itself contain a suggestion for such a modification.

This principle has been consistently upheld by the U.S. Court of Customs and Patent Appeals such as in the decision in Randol and Redford (165 USPQ 586) that:
Prior patents are references only for what they clearly disclose or suggest; it is not a proper use of a patent as a reference to modify its structure to one which prior art references do not suggest.

Also, the present invention provides for highly advantageous results. It is well known that in order to support a valid rejection the art must also suggest that it would accomplish applicant's results. This was stated by the Patent Office Board of Appeals, in the case Ex parta Tanaka, Marushima and Takahashi (174 USPQ 38) as follows:
Claims are not rejected on the ground that it would be obvious to one of ordinary skill in the art to rewire prior art devices in order to accomplish applicants' result, since there is no suggestion in prior art that such a result could be accomplished by so modifying prior art devices.

1. The method of utilizing a pneumatically operated BVM for assisting and/or enforcing the breathing process through the nose and/or mouth of a patient with mild to extremely suppressed, or without respiratory drive, though the use of a compressor with a variable positive or a variable negative pressure supplied to a rigid durable pressure chamber with BVM inside it.

2. The method of claim 1, wherein the compressor with a variable positive or a variable negative pressure pneumatically manipulates BVM inside a rigid durable pressure chamber and provides an air flow through a filtration tube inside of an electromagnetic coil, which embraced the filtration tube with a fiber rope in it and two electrically connected by a conductor ends of the fiber rope having containers with saline and hydrogen peroxide solution in each end and the fiber rope is puled by an engine, which movement is synchronized by amount of inhalations and/or exhalations by a patient and contaminated by pathogens fiber filter rope moved into a quarantine container.

3. The method of claim 1, wherein the compressor with a variable positive or a variable negative pressure connected with a rigid durable pressure chamber with BVM inside it, and said BVM provides the air flow in the filtration system to disinfect the air coming to/from the nose and/or mouth of the patient with extremely suppressed or without respiratory drive, when an energy support system feeds said compressor and an electromagnetic coil with a filter in it and which regulates frequency, amount of flow and depth of the patient's breathing while the air flow delivered to the patient is passing through an adjustable in length electromagnetic heater to deliver preheated air to the patient for some therapeutic benefits and is using the electromagnetic induction principle to convert the electric energy into heat energy alternating current of 220V, 110V or the three-phase AC 380V, 50/60 HZ into DC, then to the DC into the high frequency 1˜40 KHz high current electricity used for heating the breathing air.

4. The method of claim 1, wherein the compressor with a variable positive or a variable negative pressure connected with a rigid durable pressure chamber with BVM inside it, and said BVM provides the air flow in the filtration system to disinfect the air coining to/from the nose and/or mouth of the patient with extremely suppressed or without respiratory drive, where over a thermo filtration tube with a fiber rope inside, which is lubricated with an antibacterial liquid (for example, hydrogen peroxide) and saline on both sides of said rope, an engine moves said rope to a quarantine container for used filter-rope on one side of said rope when the both sides of the rope are connected to create a closed loop for a micro current flow induced by electromagnetic field of said coil to affect viruses infectious ability.

5. The method of claim 2, wherein the compressor with a variable positive or a variable negative pressure connected with a rigid durable pressure chamber with BVM inside it, and that provides the air flow in the filtration system through a filtration tube inside an electromagnetic coil, which embraced the filtration tube with a fiber rope in it and two electrically connected by a conductor ends of the fiber rope having containers with saline and hydrogen peroxide solution in each one and the fiber rope is puled by an engine, which movement is synchronized by a monitor that is responding to inhaled and exhaled air by a patient and said filter rope moved into a quarantine container while said air flows within an adjustable length of heating tube to and out of a patient's nose and/or mouth to implement an expiration (E) followed by an inspiration (I) phases.

6. The method of claim 1, wherein the compressor connected with a rigid durable pressure chamber with BVM inside it, and the same or another compressor provides a variable positive air pressure to a chest cuff on the patient's sternum level to complement an expiration (E) and then when followed by an inspiration (I) the pressure from said chest cuff is released.

7. The method of claim 1, wherein the compressor connected with a rigid durable pressure chamber with BVM inside it, and provided the variable negative air pressure to assist to the patient's exhalation phase, which is complimented with synchronized alternative topical stimulation of abdominal and intercostal muscles by transcutaneous electrical nerve stimulation unit (TENS) to enhance the exhalation phase of the breathing and TENS is off during an inhalation phase.

8. The method of claim 7, wherein the patient with the use of synchronized alternative topical stimulation of abdominal and intercostal muscles by TENS stimulation and variable positive air pressure to a chest cuff on the patient's sternum level to complement an expiration (E) phase and then followed by an inspiration (I) the pressure from said chest cuff is released and TENS is off.

9. A medical method for treating patients from mild to extremely suppressed or without respiratory drive who can benefit from variable negative and variable positive pressure to the thorax, the method comprising:
Implementing series of variable repeated negative pressure supplies to induce an expiration (E) immediately followed by series of variable repeated positive pressure supplies to induce an inspiration (I);
Implementing and modifying inverse ratio ventilation (IRV) by modifying the inspiratory to expiratory (I:E) ratio, with the intention to effectively assisting in breathing by increasing the mean airway pressure (MAP);
Implementing Volume Control and Pressure Control ventilation modes using one of I:E ratios of 1:2, and up as high as 1:8 with using any fractures of the numbers in said ratios to more closely mimic normal physiologic breathing;
Implementing Inverse Ratio Ventilation using one of I:E ratios of 2:1, 3:1, 4:1, and so on, up as high as 10:1 with using any fractures of the numbers in said ratios, and with inspiratory directly controlled by said expiratory times.

10. The method of claim 1, for treating patients with mild to extremely suppressed or without respiratory drive by repeatedly providing variable negative pressure while assisting a patient with the exhalation phase of the breathing to eliminate phlegm and CO2 from the patient who can benefit from a BVM complimenting inflating and deflating the ball of said BVM through changing ambient pressure outside of the ball inside said rigid durable pressure chamber that contains said ball with no mechanical contact with the ball of said BVM.

11. The method of claim 1, wherein the compressor with a variable positive or a variable negative pressure connected with a rigid durable pressure chamber with BVM inside it, and that provides the air flow from or to the patient when the air flow passing through electrically charged by PSS electrode that rotates around it's axis forced by the breathing air flow that is pushing wings that are firmly set on said electrode, clock wise or counter clockwise and brushes that are firmly attached on said electrode touching contacts inside the filter chamber and creating sparks that disable pathogens in the air while that air infected with viruses, particles and droplets of respiratory fluids containing the viruses and affect viruses in the air flow infectious ability.

12. The method of claim 1, further comprise means for changing the flow rate I/E and E/I, the composition of sweep gas, including oxygen and/or carbon dioxide ratio, lipid peroxidation (LPO) due to neutrophil-derived reactive oxygen species, and the temperature of air flowing through the filter to better assist the breathing process for the patient and to prevent or reduce the potential for causing adverse health effects or for a medically supervised increase of core temperature to eradicate viral colonies or clusters.

13. A method that provides a variable positive or a variable negative pressure from a compressor to produce directly an air flow through an inlet into a rigid durable pressure chamber (DPC) where it repositions a movable wall (MV) in the DPC that is divided by said MV, and it moves sliding valves, that are firmly attached to the MV, in a closed position and said MV delivers air to the nose and/or mouth of said patient through the filtration system during the inhalation phase of the breathing and when air pressure changed to a negative, the MV moves in the opposite direction and opens sliding valves to expel the air from said DPC and the patient according to the breathing process I/E ratio.

14. The method of claim 13, further comprised with the filtration system to disinfect the air coming to/from the nose and/or mouth of the patient with extremely suppressed or without respiratory drive, through a filtration tube inside an electromagnetic coil, which embraced the filtration tube with a fiber rope in it and two electrically connected by a conductor ends of the fiber rope having containers with saline and hydrogen peroxide solution in each one and the fiber rope is puled by an engine, which movement is synchronized by a monitor that is responding to inhaled and exhaled air by a patient and said rope moved into a quarantine container while said air flows within an adjustable length of the heating tube to and out of the patient's nose and/or mouth to implement an nspiration (I) followed by an expiration (E) phases.

15. The method of claim 14, further comprised with the filtration system to further disinfect the air coming to/from the nose and/or mouth of the patient with extremely suppressed or without respiratory drive, through a filtration method in assisting breathing that provides a filtration of the air flow from or to the patient when the air flow passing through electrically charged by PSS electrode that rotates around it's axis by the breathing air flow that is pushing wings, firmly set on said electrode, clock wise or counter clock wise and brushes that are firmly set on said electrode touching contacts inside of the filter chamber and creating sparks that disable pathogens in the air.
